(19) United States
(12) Patent Application Publication
(10) Pub. No.: US 2023/0174504 A1
(43) Pub. Date: Jun. 8, 2023

(54) PROCESS FOR PREPARATION OF 2-HYDROXY-6-((2-(1-ISOPROPYL-1H-PYRAZOL-5-YL)PYRIDIN-3-YL)METHOXY)BENZALDEHYDE

(71) Applicant: Global Blood Therapeutics, Inc., South San Francisco, CA (US)

(72) Inventors: Fang Wang, Foster City, CA (US); Youqian Deng, Sunnyvale, CA (US); Morin Mae Frick, San Mateo, CA (US); Xiang Wang, Foster City, CA (US)

(21) Appl. No.: 18/102,440

(22) Filed: Jan. 27, 2023

Related U.S. Application Data
(63) Continuation of application No. 17/843,283, filed on Jun. 17, 2022, now abandoned.
Application No. 17/843,283 is a continuation of application No. 17/520,453, filed on Nov. 5, 2021, now abandoned.
(60) Provisional application No. 63/237,780, filed on Aug. 27, 2021.
(60) Provisional application No. 63/110,826, filed on Nov. 6, 2020.

(51) Int. Cl. C07D 401/04

(52) U.S. Cl. CPC C07D 401/04; C07B 2200/13

(57) ABSTRACT

The present disclosure relates to processes for preparing a compound of Formula (I)

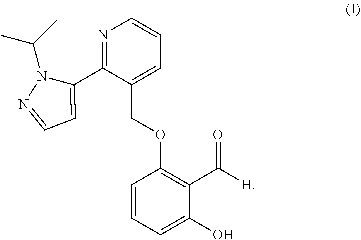

### CROSS-REFERENCE TO RELATED APPLICATIONS

This application is a continuation of U.S. application Ser. No. 17/843,283, filed Jun. 17, 2022, which is a continuation of U.S. application Ser. No. 17/520,453, filed Nov. 5, 2021, which claims the benefit under 35 U.S.C. § 119(e) of U.S. Provisional Application No. 63/110,826, filed Nov. 6, 2020, and U.S. Provisional Application No. 63/237,780, filed Aug. 27, 2021, each of which is hereby incorporated by reference in its entirety.

### BACKGROUND

2-hydroxy-6-((2-(1-isopropyl-1H-pyrazol-5-yl)pyridin-3-yl)methoxy)benzaldehyde (referred to herein as voxelotor or compound of Formula (I) and also is known as GBT440) is an approved therapeutic for the treatment of sickle cell disease.

Voxelotor is a hemoglobin S (HbS) polymerization inhibitor. By increasing the affinity of hemoglobin for oxygen, voxelotor demonstrates dose-dependent inhibition of HbS polymerization.

U.S. Pat. Nos. 9,018,210, 10,077,249, WO 2020/127924, WO/2020/128945, and Metcalf, B. et al. ACS Med. Chem. Lett. 2017, 8, 321-326, describe certain methods and intermediates for making voxelotor. There is a need for methods of making voxelotor that can ameliorate certain drawbacks encountered during scale up manufacturing of voxelotor.

### SUMMARY

Provided herein are processes for preparing a compound of Formula (I)

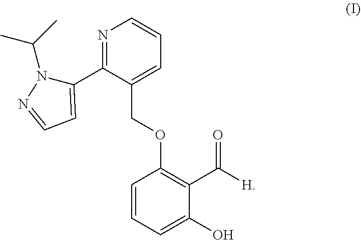

Also provided herein are intermediates, and crystalline forms thereof, useful for methods of making a compound of Formula (I).

Some embodiments provide for a crystalline form of compound (3)

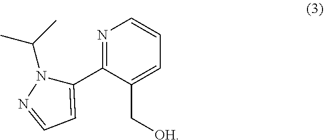 [SMILES: CC(C)n1nccc1-c1ncccc1CO]

### BRIEF DESCRIPTION OF THE DRAWINGS

Differential Scanning Calorimetry (DSC) traces are shown for an HCl salt of compound (3) (FIG. 1A) and crystalline compound (3) (FIG. 1B).

FIG. 2 shows a Dynamic Vapor Sorption (DVS) trace for crystalline compound (3).

FIG. 3 shows an X-ray powder diffraction (XRPD) spectrum for crystalline compound (3).

FIG. 4 shows a DSC trace for crystalline compound (3).

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

Reference to “about” a value or parameter herein includes (and describes) embodiments that are directed to that value or parameter per se. In other embodiments, the term “about” includes the indicated value or parameter ±5%. In certain other embodiments, the term “about” includes the indicated value or parameter ±2.5%. In certain other embodiments, the term “about” includes the indicated value or parameter ±2%. In some other embodiments, the term “about” includes the indicated value or parameter ±1%. In some other embodiments, the term “about” includes the indicated value or parameter ±0.5%. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

The terms “optional” or “optionally” means that the subsequently described event or circumstance may or may not occur, and that the description includes instances where said event or circumstance occurs and instances in which it does not.

The term “reaction conditions” is intended to refer to the physical and/or environmental conditions under which a chemical reaction proceeds. The term “under conditions sufficient to” or “under reaction conditions sufficient to” is intended to refer to the reaction conditions under which the desired chemical reaction may proceed. Examples of reaction conditions include, but are not limited to, one or more of following: reaction temperature, solvent, pH, pressure, reaction time, mole ratio of reactants, mole ratio of reagents, the presence of a base or acid, or catalyst, radiation, concentration, etc. Reaction conditions may be named after the particular chemical reaction in which the conditions are employed, such as, coupling conditions, hydrogenation conditions, acylation conditions, reduction conditions, halogenation conditions etc. Reaction conditions for most reactions are generally known to those skilled in the art or may be readily obtained from the literature. Exemplary reaction conditions sufficient for performing the chemical transformations provided herein may be found throughout the present disclosure, and in particular, the examples below. It is also contemplated that the reaction conditions may include reagents in addition to those listed in the specific reaction.

The term “reagent” refers to a substance or compound that may be added to bring about a chemical reaction.

The term “catalyst” refers to a chemical substance that enables a chemical reaction to proceed at a usually faster rate or under different conditions (such as at a lower temperature) than otherwise possible.

The term “chlorinating agent” refers to a compound that can be added to carry out a chlorination reaction. Non-limiting examples of chlorinating agents include thionyl chloride, oxalyl chloride, methanesulfonyl chloride, benzenesulfonyl chloride, toluenesulfonyl chloride, phosphorous trichloride, phosphorous pentachloride, phosphorous oxychloride, chlorine, and the like.

The terms “solvent” or “inert solvent” refer to a solvent inert under the conditions of the reaction being described in conjunction therewith.

In some embodiments, the solvent is an “organic solvent” or “inert organic solvent,” which includes, for example, benzene, toluene, acetonitrile, tetrahydrofuran (“THF”), dimethylformamide (“DMF”), chloroform, methylene chloride (or dichloromethane), diethyl ether, methanol, pyridine, and the like. Unless specified to the contrary, the solvents used in the reactions of the present disclosure are inert organic solvents, and the reactions are carried out under an inert gas, preferably nitrogen.

The term “leaving group” refers to an atom or a group of atoms that is displaced in a chemical reaction as stable species taking with it the bonding electrons. The non-limiting examples of a leaving group include, halo, methanesulfonyloxy, p-toluenesulfonyloxy, trifluoromethanesulfonyloxy, nonafluorobutanesulfonyloxy, (4-bromo-benzene)sulfonyloxy, (4-nitro-benzene)sulfonyloxy, (2-nitro-benzene)-sulfonyloxy, (4-isopropyl-benzene)sulfonyloxy, (2,4,6-tri-isopropyl-benzene)-sulfonyloxy, (2,4,6-trimethyl-benzene)sulfonyloxy, (4-tertbutyl-benzene)sulfonyloxy, benzenesulfonyloxy, (4-methoxy-benzene)sulfonyloxy, and the like.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²p, ³⁵S, ³⁶Cl and ¹²⁵I. Various isotopically labeled compounds of the present disclosure, for example, those into which radioactive isotopes such as ³H and ¹⁴C are incorporated, are provided herein. Such isotopically labeled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of compounds of Formula (I) in which from 1 to n hydrogens attached to a carbon atom is/are replaced by deuterium, in which n is the number of hydrogens in the molecule. Such compounds exhibit increased resistance to metabolism and are thus useful for increasing the half-life of any compound of Formula (I) when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example, by employing starting materials in which one or more hydrogens have been replaced by deuterium.

Deuterium labeled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compound of Formula (I).

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto.

Base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. Acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

In some cases, the “salt” of a given compound is a pharmaceutically acceptable salt. The term “pharmaceutically acceptable salt” of a given compound refers to salts that retain the biological effectiveness and properties of the given compound, and which are not biologically or otherwise undesirable. Pharmaceutically acceptable base addition salts may be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines. Specific examples of suitable amines include, by way of example only, isopropylamine, trimethyl amine, diethyl amine, tri(iso-propyl) amine, tri(n-propyl) amine, ethanolamine, diethanolamine, 2-dimethylaminoethanol, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “solid form” refers to a type of solid-state material that includes amorphous as well as crystalline forms.

The term “crystalline form” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (melting point).

The term “substantially no” or “substantially free” when qualifying any form of a compound described herein is intended to mean that no more than 0.001%; no more than 0.01%; no more than 0.1%; no more than 0.5%; no more than 1%; no more than 5%; no more than 10%; or no more than 15% of the compound is present in the designated form.

In some embodiments, the phrase “substantially shown in FIG.” as applied to an X-ray powder diffractogram is meant to include a variation of ±0.2° 2θ or ±0.1° 2θ, and as applied to DSC thermograms is meant to include a variation of ±3° C.

List of Abbreviations and Acronyms

| | |
|---|---|
| 1-IPP | 1-Isopropyl Pyrazole |
| 2-MeTHF | 2-Methyltetrahydrofuran |
| ACN | Acetonitrile |
| ADME | Absorption, Distribution, |
| | Metabolism, and Excretion |
| CC | Crash Cooling |
| CP | Crash Precipitation |
| Cₚ | Specific Heat Capacity |
| d | Day(s) |
| DCM | Dichloromethane |
| DMF | Dimethylformamide |
| DMPK | Drug Metabolism and Pharmacokinetics |
| DSC | Differential Scanning Calorimetry |
| DVS | Dynamic Vapor Sorption |
| EtOAc | Ethyl Acetate |
| EtOH | Ethanol |
| FE | Fast Evaporation |
| frz | Freezer |
| h | Hour(s) |
| HbS | Hemoglobin S |
| IPA | Isopropyl Alcohol |
| IPC | In-Process Control |
| IPOAc | Isopropyl Acetate |
| i-PrOB(pin) | 2-Isopropoxy-4,4,5,5- |
| | Tetramethyl-1,3,2-Dioxaborolane |
| LG | Leaving Group |
| MCH | Methylcyclohexane |
| MEK | Methyl Ethyl Ketone |
| MeOH | Methanol |
| min | Minute(s) |
| MTBE | Methyl tert-Butyl Ether |
| NMP | N-Methyl-2-Pyrrolidone |
| OTf | Triflate |
| Pd(Amphos)₂Cl₂ | Bis(Di-tert-Butyl(4-Dimethylaminophenyl) |
| | Phosphine)Dichloropalladium(II) |
| PET | Positron Emission Tomography |
| PTFE | Polytetrafluoroethylene |
| ref | Refrigerator |
| RH | Relative Humidity |
| RT | Room Temperature |
| SC | Slow Cooling |
| SCXRD | Single Crystal X-Ray Diffraction |
| SE | Slow Evaporation |
| SMB | Simulated Moving Bed |
| SPECT | Single-Photon Emission |
| | Computed Tomography |
| TBAB | Tetra-n-Butylammonium Bromide |
| t-Bu | tert-Butyl |
| TGA | Thermogravimetric Analysis |
| THF | Tetrahydrofuran |
| V | Volume(s) |
| VD | Vapor Diffusion |
| VS | Vapor Stress |
| v/v | Volume to Volume |
| XRPD | X-Ray Powder Diffraction |

### Processes

Provided herein are processes for preparing a compound of Formula (I). In some embodiments, provided herein is a process for preparing a compound of Formula (I)

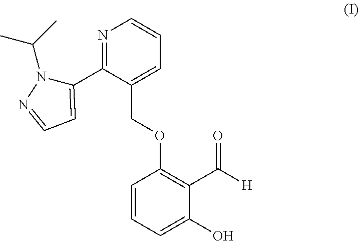 [SMILES: CC(C)n1nccc1-c1ncccc1COc1cccc(O)c1C=O]

comprising:
(i) contacting compound (1)

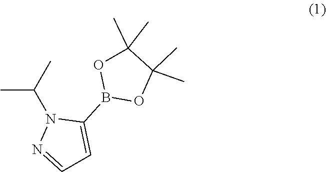 [SMILES: CC(C)n1nccc1B1OC(C)(C)C(C)(C)O1]

with compound (2)

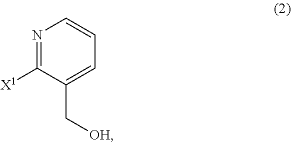

wherein X¹ is Cl, Br, I, or triflate (OTf),
in the presence of a catalyst of formula (7)

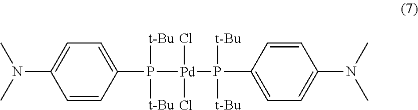

under reaction conditions sufficient to form compound (3)

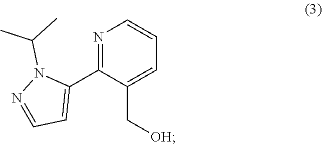 [SMILES: CC(C)n1nccc1-c1ncccc1CO]

(ii) forming a salt of compound (3);
(iii) contacting the salt of compound (3) with a base, an organic solvent, and a chlorinating agent under reaction conditions sufficient to form compound (4)

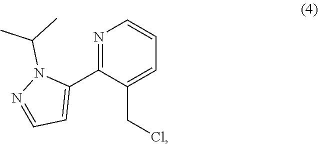 [SMILES: CC(C)n1nccc1-c1ncccc1CCl]

or a salt thereof; and
(iv) contacting compound (4), or a salt thereof, with compound (5)

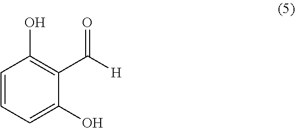

under reaction conditions sufficient to form the compound of Formula (I); and
wherein step (iii) results in substantially no formation of a solid form of compound (3).

In some embodiments, prior to contacting the salt of compound (3) with a chlorinating agent, substantially no formation of a solid form of compound (3) occurs.

In some embodiments, the base of step (iii) (i.e. contacting the salt of compound (3) with a base, an organic solvent, and a chlorinating agent under reaction conditions sufficient to form compound (4)) comprises sodium bicarbonate, the organic solvent of step (iii) comprises dichloromethane, and the chlorinating agent of step (iii) comprises SOCl₂.

In some embodiments, the salt of compound (3) is contacted with an organic solvent, followed by a base, and followed by a chlorinating agent.

In some embodiments, provided herein is a process for preparing a compound of Formula (I)

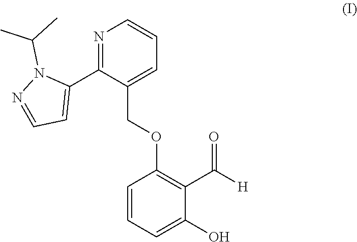 [SMILES: CC(C)n1nccc1-c1ncccc1COc1cccc(O)c1C=O]

comprising:
(i) contacting compound (1)

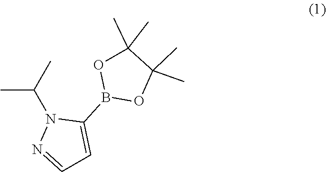 [SMILES: CC(C)n1nccc1B1OC(C)(C)C(C)(C)O1]

with compound (2)

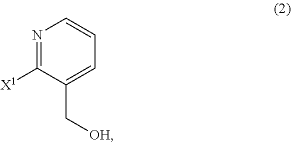

wherein X¹ is Cl, Br, I, or triflate (OTf),
in the presence of a catalyst of formula (7)

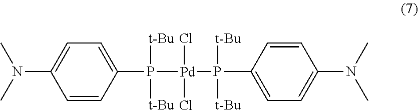

under reaction conditions sufficient to form compound (3)

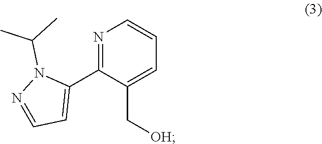 [SMILES: CC(C)n1nccc1-c1ncccc1CO]

(ii) forming a salt of compound (3);
(iii) contacting the salt of compound (3) with an organic solvent to form a mixture;
(iv) contacting the mixture with a base to form a solution of compound (3);
(v) contacting the solution of compound (3) with a chlorinating agent under reaction conditions sufficient to form compound (4)

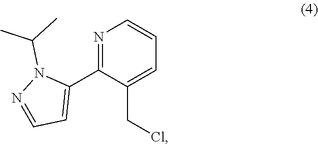 [SMILES: CC(C)n1nccc1-c1ncccc1CCl]

or a salt thereof; and
(vi) contacting compound (4), or a salt thereof, with compound (5)

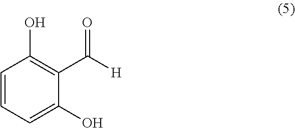

under reaction conditions sufficient to form the compound of Formula (I).

In some embodiments, the organic solvent of step (iii) comprises dichloromethane, the base of step (iv) comprises sodium bicarbonate, and the chlorinating agent of step (v) comprises SOCl₂.

In some embodiments, provided herein is a process for preparing a compound of Formula (I)

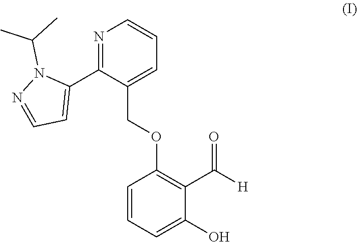 [SMILES: CC(C)n1nccc1-c1ncccc1COc1cccc(O)c1C=O]

comprising:
(i) contacting compound (1)

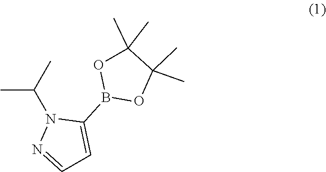 [SMILES: CC(C)n1nccc1B1OC(C)(C)C(C)(C)O1]

with compound (2)

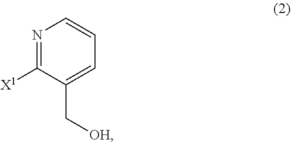

wherein X¹ is Cl, Br, I, or triflate (OTf),
in the presence of a catalyst of formula (7)

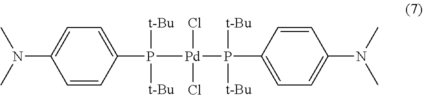

under reaction conditions sufficient to form compound (3)

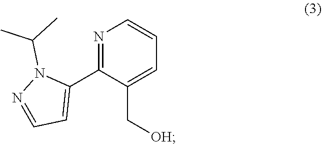 [SMILES: CC(C)n1nccc1-c1ncccc1CO]

(ii) contacting compound (3) with an organic solvent and a chlorinating agent under reaction conditions sufficient to form compound (4)

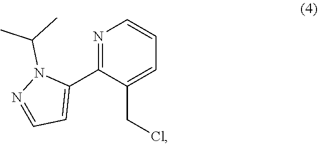 [SMILES: CC(C)n1nccc1-c1ncccc1CCl]

or a salt thereof; and
(iii) contacting compound (4), or a salt thereof, with compound (5)

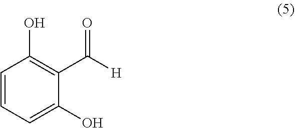

under reaction conditions sufficient to form the compound of Formula (I).

In some embodiments, the reaction conditions in step (i) (i.e. contacting compound (1) with compound (2)) comprise 2-methyltetrahydrofuran as a solvent.

In some embodiments, the reaction conditions in step (i) (i.e. contacting compound (1) with compound (2)) comprise a temperature of about 70° C. to about 80° C. In some embodiments, the reaction conditions in step (i) comprise a temperature of about 72° C. to about 77° C. In some embodiments, the reaction conditions in step (i) comprise a temperature of about 75° C.

In some embodiments, the reaction conditions in step (i) (i.e. contacting compound (1) with compound (2)) comprise about 1.0 equivalent to 3.0 equivalents of a base relative to equivalents of compound (2). In some embodiments, the reaction conditions in step (i) comprise about 2 equivalents of a base relative to equivalents of compound (2). In some embodiments, the reaction conditions in step (i) comprise about 2.5 equivalents of a base relative to equivalents of compound (2). In some embodiments, the reaction conditions in step (i) comprise about 3 equivalents of a base relative to equivalents of compound (2). In such embodiments, the base of step (i) is sodium bicarbonate.

In some embodiments, the reaction conditions in step (i) (i.e. contacting compound (1) with compound (2)) comprise about 1.0 equivalent to 2.0 equivalents of compound (1) relative to equivalents of compound (2). In some embodiments, the reaction conditions in step (i) comprise about 1.0 equivalent to 1.5 equivalents of compound (1) relative to equivalents of compound (2). In some embodiments, the reaction conditions in step (i) comprise about 1.5 equivalents of compound (1) relative to equivalents of compound (2). In some embodiments, the reaction conditions in step (i) comprise about 1.3 equivalents of compound (1) relative to equivalents of compound (2).

In some embodiments, the reaction conditions in step (i) (i.e. contacting compound (1) with compound (2)) comprise about 0.001 equivalents to about 0.005 equivalents of the catalyst relative to equivalents of compound (2). In some embodiments, the reaction conditions in step (i) comprise about 0.005 equivalents of the catalyst relative to equivalents of compound (2). In some embodiments, the reaction conditions in step (i) with compound (2)) comprise about 0.004 equivalents of the catalyst relative to equivalents of compound (2). In some embodiments, the reaction conditions in step (i) comprise about 0.003 equivalents of the catalyst relative to equivalents of compound (2). In some embodiments, the reaction conditions in step (i) comprise about 0.002 equivalents of the catalyst relative to equivalents of compound (2). In some embodiments, the reaction conditions in step (i) comprise about 0.001 equivalents of the catalyst relative to equivalents of compound (2).

In some embodiments, the organic solvent of step (ii) (i.e. contacting compound (3) with an organic solvent and a chlorinating agent under reaction conditions sufficient to form compound (4)) comprises dichloromethane, and the chlorinating agent of step (ii) comprises SOCl₂.

In some embodiments, compound (3) is contacted with an organic solvent, followed by a chlorinating agent. In some embodiments, compound (3) is concurrently contacted with the organic solvent and the chlorinating agent. In some embodiments, the organic solvent and the chlorinating agent are added sequentially (in any order) to compound (3).

In some embodiments, provided herein is a process for preparing a compound of Formula (I)

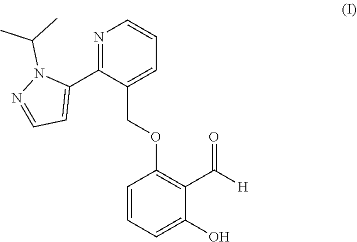 [SMILES: CC(C)n1nccc1-c1ncccc1COc1cccc(O)c1C=O]

comprising
(i) contacting about 1.3 equivalents of compound (1)

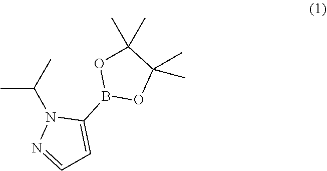 [SMILES: CC(C)n1nccc1B1OC(C)(C)C(C)(C)O1]

with 1 equivalent of compound (2)

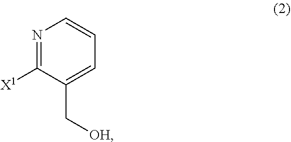

wherein X¹ is Cl, Br, I, or OTf,
in the presence of about 0.003 equivalents of a catalyst of formula (7)

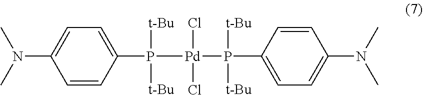

relative to equivalents of compound (2),
and about 2.5 equivalents of sodium bicarbonate relative to equivalents of compound (2),
in 2-methyltetrahydrofuran as a solvent,
at a temperature of about 70° C. to about 80° C. to form compound (3)

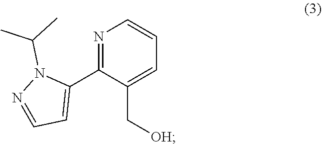 [SMILES: CC(C)n1nccc1-c1ncccc1CO]

(ii) forming a salt of compound (3);
(iii) contacting the salt of compound (3) with a base, an organic solvent, and a chlorinating agent under reaction conditions sufficient to form compound (4)

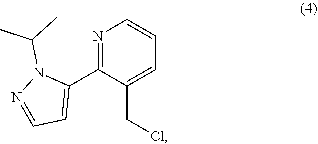 [SMILES: CC(C)n1nccc1-c1ncccc1CCl]

or a salt thereof; and
(iv) contacting compound (4), or a salt thereof, with compound (5)

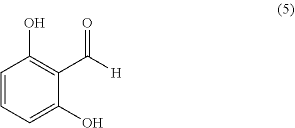

under reaction conditions sufficient to form the compound of Formula (I).

Some embodiments further comprise crystallizing the compound of Formula (I) to obtain a crystalline ansolvate of the compound of Formula (I) characterized by an X-ray powder diffractogram comprising the following peaks: 13.37°, 14.37°, 19.95° and 23.92° 2θ, each ±0.2 ° 2θ, as determined on a diffractometer using Cu-Kα radiation. This crystalline ansolvate is known as Form II of a compound of Formula (I).

In some embodiments, the crystalline ansolvate of the compound of Formula (I) is characterized by an endothermic peak at 97±2° C. as measured by differential scanning calorimetry.

In some embodiments, crystallizing comprises contacting the compound of Formula (I) with methyl tert-butyl ether (MTBE) and n-heptane.

In some embodiments, Form II, and other forms of a compound of Formula (I), including but not limited to Form I and Material N, can be prepared according to methods described in U.S. Pat. No. 9,447,071. XRPD patterns for such forms can be carried out according to methods described in U.S. Pat. No. 9,447,071.

In some embodiments, the crystalline ansolvate of the compound of Formula (I) substantially free of other ansolvate polymorphs of compound of Formula (I).

Some embodiments provided herein further comprise isolating the compound of Formula (I) by adding 10% brine as an antisolvent.

In some embodiments, adding 10% brine as an antisolvent provides increased yield of compound of Formula (I) compared to adding water as an antisolvent.

In some embodiments, provided herein is a process for preparing a compound of Formula (I)

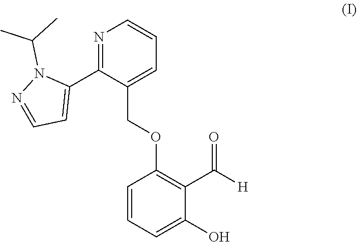 [SMILES: CC(C)n1nccc1-c1ncccc1COc1cccc(O)c1C=O]

comprising
(i) contacting about 1.3 equivalents of compound (1)

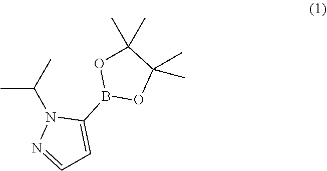 [SMILES: CC(C)n1nccc1B1OC(C)(C)C(C)(C)O1]

with 1 equivalent of compound (2)

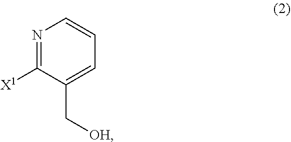

wherein X¹ is Cl, Br, I, or OTf,
in the presence of about 0.003 equivalents of a catalyst of formula (7)

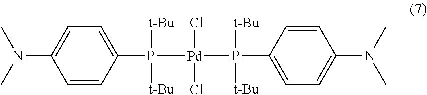

relative to equivalents of compound (2),
and about 2.5 equivalents of sodium bicarbonate relative to equivalents of compound (2),
in 2-methyltetrahydrofuran as a solvent,
at a temperature of about 70° C. to about 80° C. to form compound (3)

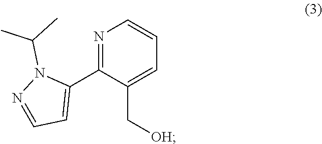 [SMILES: CC(C)n1nccc1-c1ncccc1CO]

(ii) forming a salt of compound (3);
(iii) contacting the salt of compound (3) with a base, an organic solvent, and a chlorinating agent under reaction conditions sufficient to form compound (4)

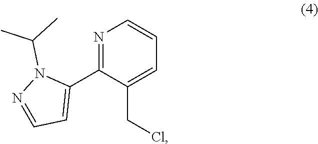 [SMILES: CC(C)n1nccc1-c1ncccc1CCl]

or a salt thereof;
(iv) contacting compound (4), or a salt thereof, with compound (5)

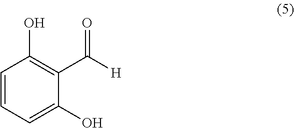

under reaction conditions sufficient to form the compound of Formula (I); and (vi) contacting compound of Formula (I) with methyl tert-butyl ether and n-heptane under conditions sufficient to form a crystalline ansolvate of the compound of Formula (I) characterized by an X-ray powder diffractogram comprising the following peaks: 13.37°, 14.37°, 19.95° and 23.92° 2θ, each ±0.2° 2θ, as determined on a diffractometer using Cu-Kα radiation.

In some embodiments, step (i) (i.e. contacting about 1.3 equivalents of compound (1) with 1 equivalent compound (2)) is performed at a temperature of about 75° C.

In some embodiments, the base of step (iii) (i.e. contacting the salt of compound (3) with a base, an organic solvent, and a chlorinating agent under reaction conditions sufficient to form compound (4)) comprises sodium bicarbonate, the organic solvent of step (iii) comprises dichloromethane, and the chlorinating agent of step (iii) comprises SOCl₂.

In some embodiments, the salt of compound (3) is contacted with an organic solvent, followed by a base, and followed by a chlorinating agent. In some embodiments, the salt of compound (3) is concurrently contacted with the base, the organic solvent, and the chlorinating agent. In some embodiments, the base, the organic solvent, and the chlorinating agent are added sequentially (in any order) to the salt of compound (3).

In some embodiments, provided herein is a process for preparing a compound of Formula (I)

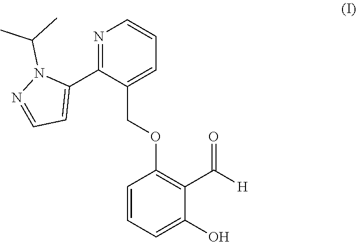 [SMILES: CC(C)n1nccc1-c1ncccc1COc1cccc(O)c1C=O]

comprising
(i) contacting 1-isopropyl pyrazole with 2-isopropoxy-4,4,5,5-tetramethyl-1,3,2-dioxaborolane under conditions sufficient to form compound (1)

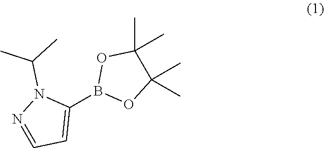 [SMILES: CC(C)n1nccc1B1OC(C)(C)C(C)(C)O1]

(ii) contacting about 1.3 equivalents of compound (1) with 1 equivalent of compound (2)

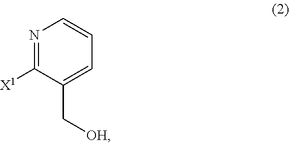

wherein X¹ is Cl, Br, I, or OTf,
in the presence of about 0.003 equivalents of a catalyst of formula (7)

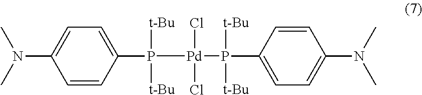

relative to equivalents of compound (2),
and about 2.5 equivalents of sodium bicarbonate relative to equivalents of compound (2),
in 2-methyltetrahydrofuran as a solvent,
at a temperature of about 70° C. to about 80° C. to form compound (3)

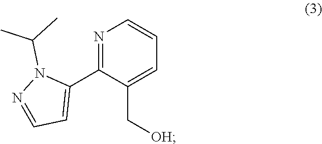 [SMILES: CC(C)n1nccc1-c1ncccc1CO]

(iii) forming a salt of compound (3);
(iv) contacting the salt of compound (3) with a base, an organic solvent, and a chlorinating agent under reaction conditions sufficient to form compound (4)

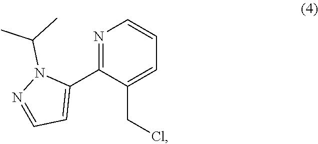 [SMILES: CC(C)n1nccc1-c1ncccc1CCl]

or a salt thereof;
(v) contacting compound (4), or a salt thereof, with compound (5)

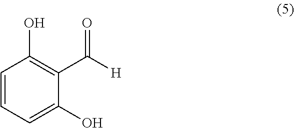

under reaction conditions sufficient to form the compound of Formula (I); and (vi) contacting compound of Formula (I) with methyl tert-butyl ether and n-heptane under conditions sufficient to form a crystalline ansolvate of the compound of Formula (I) characterized by an X-ray powder diffractogram comprising the following peaks: 13.37°, 14.37°, 19.95° and 23.92° 2θ, each ±0.2° 2θ, as determined on a diffractometer using Cu-Kα radiation.

In some embodiments, the conditions of step (i) (i.e. contacting 1-isopropyl pyrazole with 2-isopropoxy-4,4,5,5-tetramethyl-1,3,2-dioxaborolane) comprise a base. In some embodiments, the base is n-butyllithium. In some embodiments, the conditions of step (i) comprise 2-methyltetrahydrofuran as a solvent.

In some embodiments, provided herein is a process for preparing a compound of Formula (I)

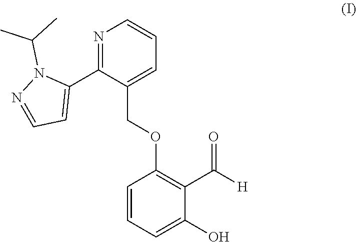 [SMILES: CC(C)n1nccc1-c1ncccc1COc1cccc(O)c1C=O]

comprising
(i) contacting 1H-pyrazole with compound (6)

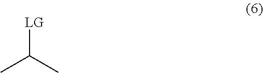

wherein LG is a leaving group,
under conditions sufficient to form 1-isopropyl pyrazole;
(ii) contacting 1-isopropyl pyrazole with 2-isopropoxy-4,4,5,5-tetramethyl-1,3,2-dioxaborolane under conditions sufficient to form compound (1)

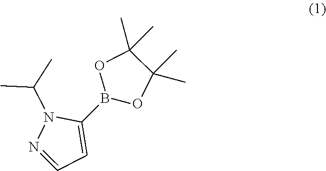 [SMILES: CC(C)n1nccc1B1OC(C)(C)C(C)(C)O1]

(iii) contacting about 1.3 equivalents of compound (1) with 1 equivalent of compound (2)

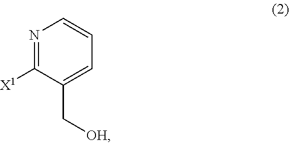

wherein X¹ is Cl, Br, I, or OTf,
in the presence of about 0.003 equivalents of a catalyst of formula (7)

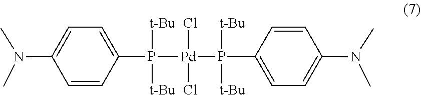

relative to equivalents of compound (2),
and about 2.5 equivalents of sodium bicarbonate relative to equivalents of compound (2),
in 2-methyltetrahydrofuran as a solvent,
at a temperature of about 70° C. to about 80° C. to form compound (3)

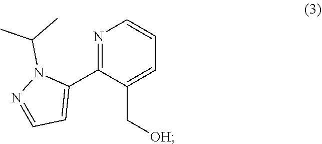 [SMILES: CC(C)n1nccc1-c1ncccc1CO]

(iv) forming a salt of compound (3);
(v) contacting the salt of compound (3) with a base, an organic solvent, and a chlorinating agent under reaction conditions sufficient to form compound (4)

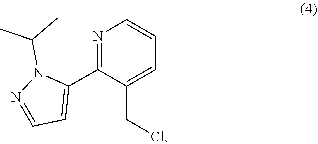 [SMILES: CC(C)n1nccc1-c1ncccc1CCl]

or a salt thereof;
(vi) contacting compound (4), or a salt thereof, with compound (5)

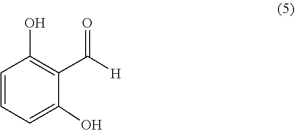

under reaction conditions sufficient to form the compound of Formula (I); and (vi) contacting compound of Formula (I) with methyl tert-butyl ether and n-heptane under conditions sufficient to form a crystalline ansolvate of the compound of Formula (I) characterized by an X-ray powder diffractogram comprising the following peaks: 13.37°, 14.37°, 19.95° and 23.92° 2θ, each ±0.2° 2θ, as determined on a diffractometer using Cu-Kα radiation.

In some embodiments, LG is halo. In some embodiments, LG is Cl or Br. In some embodiments, LG is Br.

In some embodiments, the conditions of step (i) (i.e. contacting 1H-pyrazole with compound (6)) comprise tetra-n-butylammonium bromide. In some embodiments, the reaction conditions of step (i) comprise water.

In some embodiments, LG is Br, and the conditions of step (i) (i.e. contacting 1H-pyrazole with compound (6)) comprise water and tetra-n-butylammonium bromide.

In some embodiments, the salt of compound (3) is a hydrochloric acid salt. In some embodiments, forming a salt of compound (3) comprises contacting compound (3) with HCl.

In some embodiments, the salt of compound (4) is a hydrochloric acid salt. In some embodiments, the salt of compound (4) is a monohydrochloric acid salt. In some embodiments, the salt of compound (4) is a bishydrochloric acid salt.

In some embodiments, forming a salt of compound (3) comprises contacting compound (3) with HCl.

In some embodiments, X¹ is Cl or Br. In some embodiments, X¹ is Cl.

In some embodiments, reaction conditions for contacting compound (4), or a salt thereof, with compound (5)) comprise N-methyl-2-pyrrolidone (NMP), sodium bicarbonate, and NaI.

The use of palladium catalysts can be expensive and lead to toxic waste. Thus, the use of efficient palladium catalysts that can reduce catalyst loading and/or provide complete conversion is desirable. Coupling compound (1) with compound (2) in the presence of Pd(Amphos)₂Cl₂ (Bis(di-tert-butyl(4-dimethylaminophenyl)phosphine)dichloropalladium(II) and referred to herein as compound (7) or catalyst of formula (7), wherein t-Bu refers to tert-butyl) provides high yields (e.g. greater than 95%) of compound (3) with low catalyst loading. It is contemplated that a combination of reaction conditions provides improved yields of compound (3), such as the relative equivalents of a compound (1) to compound (2), the amount of base, the amount of catalyst, the choice of solvent, and the reaction temperature.

Additionally, it is contemplated that methods described herein provide improved methods of making a compound of formula (I) that avoids formation of a solid form of compound (3).

### Intermediates, Crystalline Forms, and Methods of Making Thereof

Also provided herein is a crystalline form of compound (3)

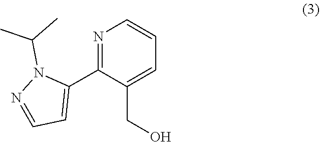 [SMILES: CC(C)n1nccc1-c1ncccc1CO]

characterized by an X-ray powder diffractogram comprising the following peaks: 14.79°, 22.67°, and 24.44° 2θ, each ±0.2° 2θ, as determined on a diffractometer using Cu-Kα radiation.

In some embodiments of processes described herein, compound (3) is isolated in a crystalline form characterized by an X-ray powder diffractogram comprising the following peaks: 14.79°, 22.67°, and 24.44° 2θ, each ±0.2° 2θ, as determined on a diffractometer using Cu-Kα radiation.

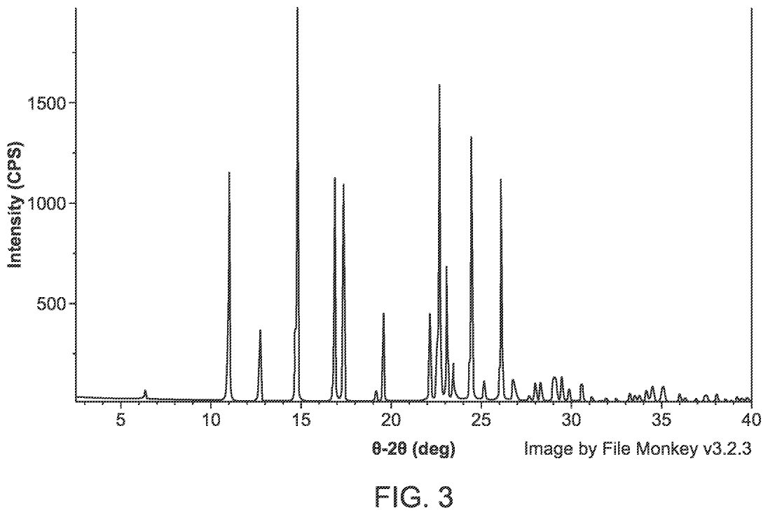

In some embodiments, the diffractogram further comprises peaks at 11.02°, 16.88°, 17.34°, and 26.09° 2θ, each ±0.2° 2θ. In some embodiments, the crystalline form of compound (3) is characterized by an X-ray powder diffractogram as substantially shown in FIG. 3.

In some embodiments, the diffractogram further comprises peaks at 12.73°, 19.57°, 22.16°, and 23.08° 2θ, each ±0.2° 2θ.

In some embodiments, the diffractogram comprises the following peaks: 11.02°, 12.73°, 14.79°, 16.88°, 17.34°, 19.57°, 22.16°, 22.67°, 23.08°, 24.44°, and 26.09° 2θ, each ±0.2 °2θ, as determined on a diffractometer using Cu-Kα radiation.

In some embodiments, the crystalline form of compound (3) is characterized by a differential scanning calorimetry (DSC) curve comprising an endotherm peak at about 80° C. In some embodiments, the crystalline form of compound (3) is characterized by a DSC curve as substantially shown in FIG. 1B.

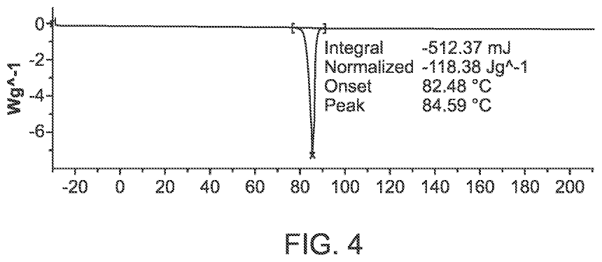

In some embodiments, the crystalline form of compound (3) is characterized by a differential scanning calorimetry (DSC) curve comprising an endotherm at about 82° C. (onset temperature). In some embodiments, the crystalline form of compound (3) is characterized by a DSC curve as substantially shown in FIG. 4.

Further provided is a method for preparing the crystalline form of compound (3), the method comprising contacting a salt of compound (3) with aqueous sodium bicarbonate. In some of such embodiments, the mixture is optionally filtered and dried to obtain crystalline compound (3).

In some embodiments, provided herein is a process for preparing a compound of (3) comprising contacting about 1.3 equivalents of compound (1)

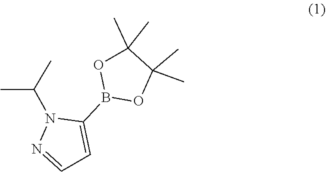 [SMILES: CC(C)n1nccc1B1OC(C)(C)C(C)(C)O1]

with 1 equivalent of compound (2)

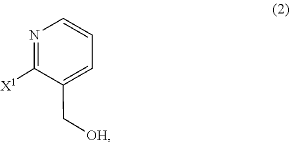

wherein X¹ is Cl, Br, I, or OTf,
in the presence of about 0.003 equivalents of a catalyst of formula (7)

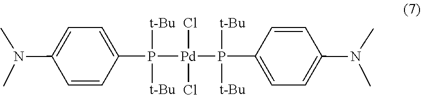

relative to equivalents of compound (2),
and about 2.5 equivalents of sodium bicarbonate relative to equivalents of compound (2),
in 2-methyltetrahydrofuran as a solvent,
at a temperature of about 70° C. to about 80° C. to form compound (3).

In some embodiments, contacting about 1.3 equivalents of compound (1) with 1 equivalent of compound (2) is performed at a temperature of about 75° C.

In some embodiments, provided herein is a process for preparing a compound of (3) comprising
(i) contacting 1-isopropyl pyrazole with 2-isopropoxy-4,4,5,5-tetramethyl-1,3,2-dioxaborolane under conditions sufficient to form compound (1)

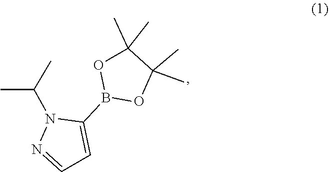 [SMILES: CC(C)n1nccc1B1OC(C)(C)C(C)(C)O1]

(ii) contacting about 1.3 equivalents of compound (1) with 1 equivalent of compound (2)

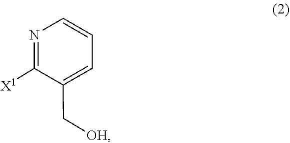

wherein X¹ is Cl, Br, I, or OTf,
in the presence of about 0.003 equivalents of a catalyst of formula (7)

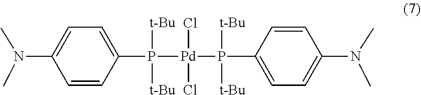

relative to equivalents of compound (2),
and about 2.5 equivalents of sodium bicarbonate relative to equivalents of compound (2),
in 2-methyltetrahydrofuran as a solvent,
at a temperature of about 70° C. to about 80° C. to form compound (3).

In some embodiments, the conditions of step (i) (i.e. contacting 1-isopropyl pyrazole with 2-isopropoxy-4,4,5,5-tetramethyl-1,3,2-dioxaborolane) comprise a base. In some embodiments, the base is n-butyllithium. In some embodiments, the conditions of step (i) comprise 2-methyltetrahydrofuran as a solvent.

In some embodiments, step (ii) (i.e. contacting about 1.3 equivalents of compound (1) with 1 equivalent compound (2)) is performed at a temperature of about 75° C.

In some embodiments, provided herein is a process for preparing a compound of (3)

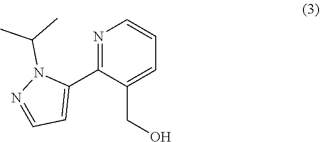 [SMILES: CC(C)n1nccc1-c1ncccc1CO]

comprising
(i) contacting 1H-pyrazole with compound (6)

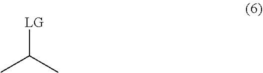

wherein LG is a leaving group,
under conditions sufficient to form 1-isopropyl pyrazole; and
(ii) contacting 1-isopropyl pyrazole with 2-isopropoxy-4,4,5,5-tetramethyl-1,3,2-dioxaborolane under conditions sufficient to form compound (1)

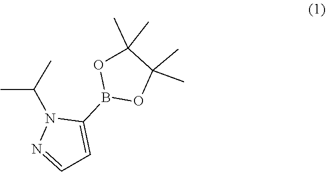 [SMILES: CC(C)n1nccc1B1OC(C)(C)C(C)(C)O1]

(iii) contacting about 1.3 equivalents of compound (1) with 1 equivalent of compound (2)

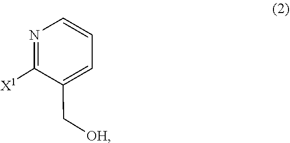

wherein X¹ is Cl, Br, I, or OTf,
in the presence of about 0.003 equivalents of a catalyst of formula (7)

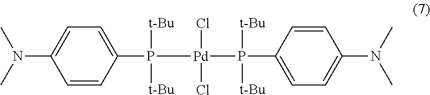

relative to equivalents of compound (2),
and about 2.5 equivalents of sodium bicarbonate relative to equivalents of compound (2),
in 2-methyltetrahydrofuran as a solvent,
at a temperature of about 70° C. to about 80° C. to form compound (3).

In some embodiments, LG is halo. In some embodiments, LG is Cl or Br. In some embodiments, LG is Br.

In some embodiments, the conditions of step (i) (i.e. contacting 1H-pyrazole with compound (6)) comprise tetra-n-butylammonium bromide. In some embodiments, the reaction conditions of step (i) comprise water. In some embodiments, LG is Br, and the conditions of step (i) (i.e. contacting 1H-pyrazole with compound (6)) comprise water and tetra-n-butylammonium bromide.

In some embodiments, the conditions of step (ii) (i.e. contacting 1-isopropyl pyrazole with 2-isopropoxy-4,4,5,5-tetramethyl-1,3,2-dioxaborolane) comprise a base. In some embodiments, the base is n-butyllithium. In some embodiments, the conditions of step (ii) comprise 2-methyltetrahydrofuran as a solvent.

In some embodiments, step (iii) (i.e. contacting about 1.3 equivalents of compound (1) with 1 equivalent compound (2)) is performed at a temperature of about 75° C.

The starting materials and reagents for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

In the scheme and examples provided below it may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium, and low pressure liquid chromatography methods and apparatus; small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like.

Selection of appropriate methods of separation depends on the nature of the materials involved. For example, boiling point, and molecular weight in distillation and sublimation, presence or absence of polar functional groups in chromatography, stability of materials in acidic and basic media in multiphase extraction, and the like. One skilled in the art will apply techniques most likely to achieve the desired separation.

Scheme 1 below shows an embodiment of the general method for the synthesis of voxelotor described herein.

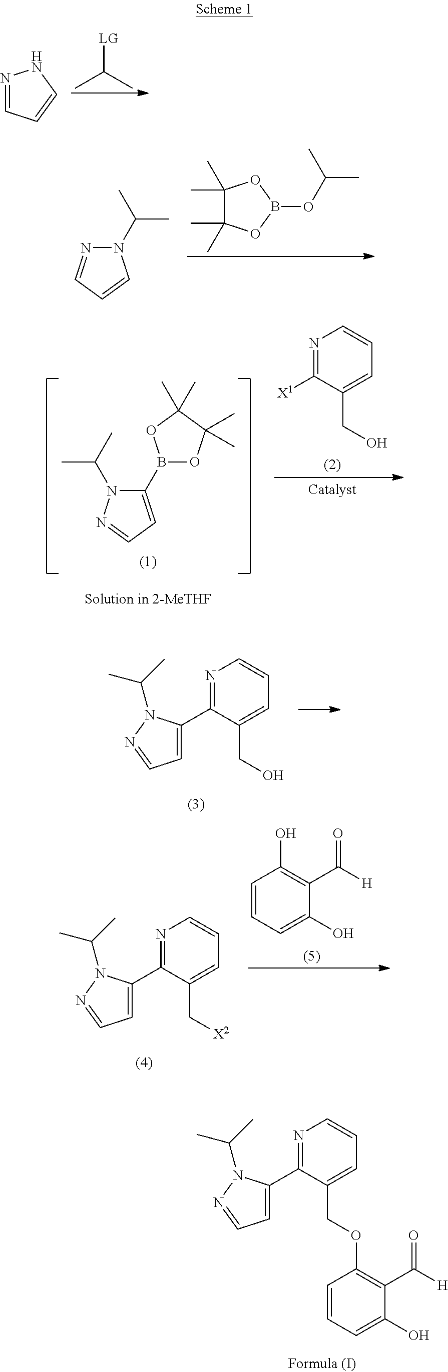

In some embodiments, X¹ and X² are each independently Cl, Br, I, or triflate (—OTf). In some embodiments, X¹ is Cl or Br. In some embodiments, X² is Cl or Br. In some embodiments, X¹ is Cl. In some embodiments, X² is Cl. In some embodiments, X¹ is Cl and X² is Cl. In some embodiments, LG is halo. In some embodiments, LG is Br. In some embodiments, LG is Br, X¹ is Cl, and X² is Cl.

In some embodiments, isopropyl pyrazole may be prepared using conventional techniques and then converted to boronate (1), which can be used as a solution in 2-methyl-tetrahydrofuran (2-Me THF). Other suitable solvents may be used for the preparation of the boronate. The reaction of boronate (1) with compound (2) may be carried out in the presence of a suitable catalyst, such as Pd(Amphos)₂Cl₂, to provide compound (3). Conversion of compound (3) to compound (4) in the presence of a base, followed by coupling compound (4) with compound (5) provides the compound of Formula (I).

Compound of Formula (I) can be obtained in substantially high purity by using 10% brine as an anti-solvent during the work-up of the reaction as described herein and in the Examples below. In some embodiments, a crystalline form of compound of Formula (I) can be obtained, such as by crystallization (e.g., from a mixture of MTBE-heptane).

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

The compounds including intermediates may be prepared using methods disclosed herein and routine modifications thereof which will be apparent given the disclosure herein and methods well known in the art. Conventional and well-known synthetic methods may be used in addition to the teachings herein. The synthesis of compounds described herein, may be accomplished as described in the following examples. If available, reagents may be purchased commercially, e.g. from Sigma Aldrich or other chemical suppliers. Unless otherwise noted, the starting materials for the following reactions may be obtained from commercial sources.

### Example 1: Preparation of Compound (1)

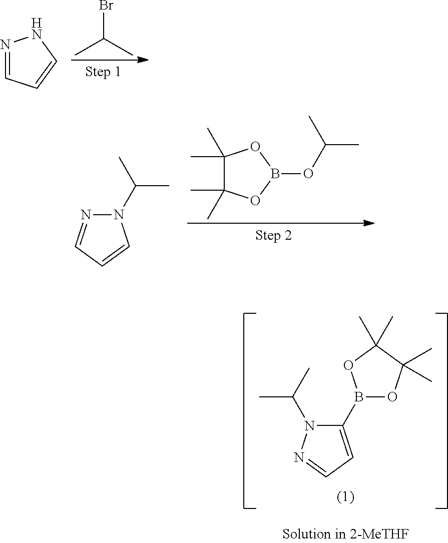

### Step 1

Water, tetra-n-butylammonium bromide (TBAB), and pyrazole (1 equivalent) were charged to a reactor, and the contents agitated until a clear solution is formed. 2-Bromopropane (1.5 equivalents) and aqueous NaOH (1.7 equivalents) were charged to the reactor, and the contents heated to about 55° C. with agitation until reaction completion. The contents of reactor A were cooled, and the layers then settled. The lower aqueous layer was removed and discarded.

The contents of reactor A were agitated and heated to distill 2-bromopropane under atmospheric pressure. The contents of reactor A were then further heated to distill 1-isopropyl pyrazole/water under reduced pressure. The contents of reactor A were heated to distill 1-isopropyl pyrazole (1-IPP) under reduced pressure.

### Step 2

1-IPP (1 equivalent) and 2-methyltetrahydrofuran (2-MeTHF) were charged to a reactor, and the contents were agitated and cooled to −15° C. 25% n-Butyllithium in heptane solution (1.2 equivalents) and 2-isopropoxy-4,4,5,5-tetramethyl-1,3,2-dioxaborolane (i-PrOB (pin), about 1 equivalent) were charged to the reactor while maintaining temperature. The contents of the reactor were agitated and sampled for reaction completion.

The contents of reactor A were heated to 15° C., and then glacial acetic acid was charged while maintaining temperature. 12.5% NaCl solution was charged to the reactor, and the contents were agitated. The contents of the reactor settled, and the bottom aqueous layer was removed. Water was charged to the reactor, and the contents were agitated. The contents of reactor A settled, and the lower aqueous layer was removed. The contents of the reactor were concentrated under reduced pressure to provide the compound (1) solution.

### Example 2: Preparation of a Hydrochloric Acid Salt of Compound (3)

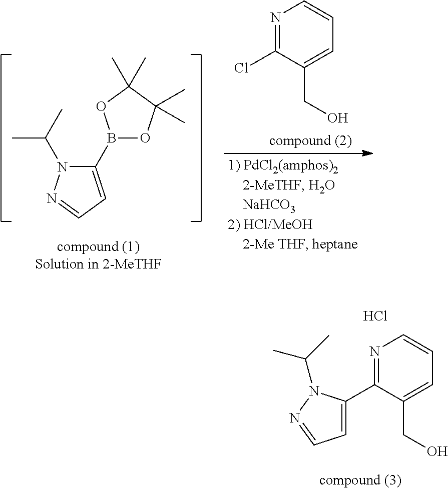

A mixture of compound (2) (1 equivalent), sodium bicarbonate (2.5 equivalents), Pd(Amphos)₂Cl₂ catalyst (0.003 equivalents), compound (1) solution in 2-MeTHF (1.3 equivalents), and water was agitated and degassed. The resulting mixture was heated (at a temperature of about 70 to about 80° C.) until reaction was complete.

The reactor content was washed with aqueous NaCl solution. The organic layer was diluted with heptane and concentrated repeatedly. 2-MeTHF and heptane were charged to the reactor, and the contents were then transferred and filtered through charcoal into a clean reactor.

Seeds of a hydrochloric acid salt of compound (3), made according to methods described herein or as known in the art, and methanolic HCl were charged to the reactor and the contents were cooled and agitated while maintaining temperature. The contents of the reactor were isolated, and the cake was washed with 2-MeTHF and dried under reduced pressure to provide a hydrochloric acid salt of compound (3).

### Example 3: Preparation of Compound (3) and Characterization of Crystalline Compound (3)

A hydrochloric acid salt of compound (3) was charged to a reactor, followed by addition of water (2.5V). The resulting solution was passed through a charcoal filter and back-added to the reactor. Aqueous sodium bicarbonate (8%, 5V) was then charged to the filtrate over 1 h at 15° C. At the end of the sodium bicarbonate addition (pH˜8), the reactor contents formed a white slurry. The white solids were isolated, providing a crystalline compound (3).

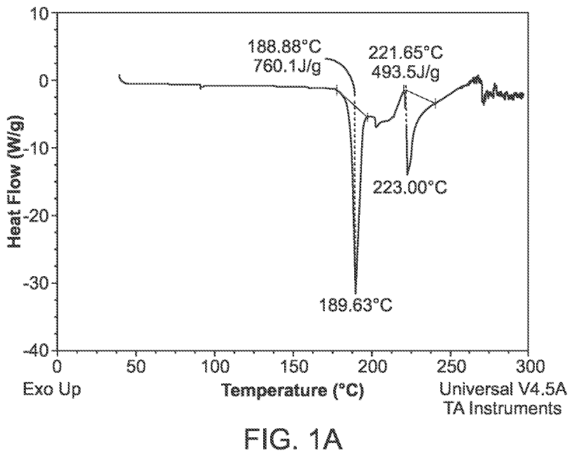

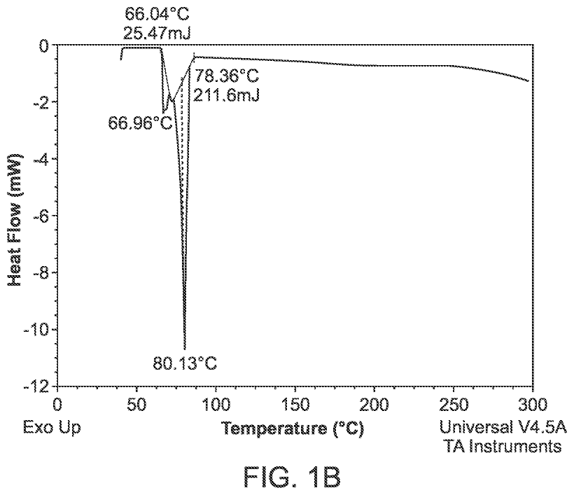

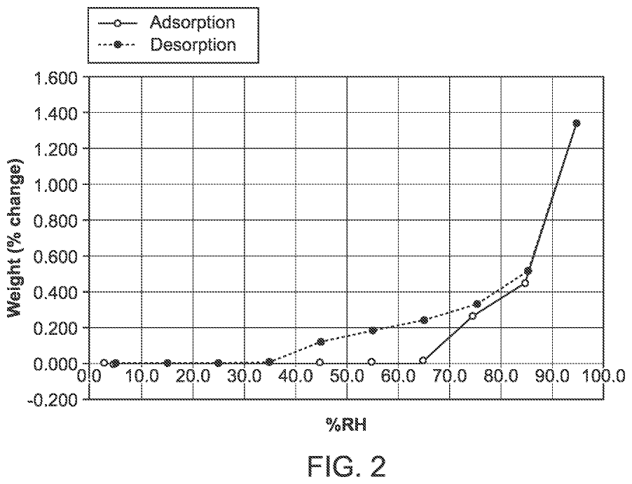

A crystalline compound (3) (compound (3) Form A) was analyzed by XRPD (FIG. 3), DSC (FIG. 1B and FIG. 4), DVS (FIG. 2), and thermogravimetry (TGA).

XRPD patterns for compound (3) were collected with a PANalytical X′Pert PRO MPD diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640e or 640f) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and an antiscatter knife edge were used to minimize the background generated by air. Soller slits for the incident and diffracted beams were used to minimize broadening from axial divergence. Diffraction patterns were collected using a scanning position-sensitive detector (X′Celerator) located 240 mm from the specimen and Data Collector software v. 5.5.

DSC was performed using a Mettler-Toledo DSC3+ differential scanning calorimeter. A tau lag adjustment was performed with indium, tin, and zinc. The temperature and enthalpy were adjusted with octane, phenyl salicylate, indium, tin and zinc. The adjustment was then verified with octane, phenyl salicylate, indium, tin, and zinc. The sample was placed into a hermetically sealed aluminum DSC pan, the weight was accurately recorded, and the sample was inserted into the DSC cell. A weighed aluminum pan configured as the sample pan was placed on the reference side of the cell. The pan lid was pierced prior to sample analysis. For standard DSC analysis, the sample was analyzed from −25° C. to 250° C. at 10° C./min.

Vapor sorption data were collected on a SGA-100 Symmetric Vapor Sorption Analyzer. Sorption and desorption data were collected over a range from 5% to 95% relative humidity (“RH”) at 10% RH increments under a nitrogen purge. The equilibrium criterion used for analysis was less than 0.0100% weight change in 5 minutes with a maximum equilibration time of 3 hours.

Thermogravimetric analysis was performed using a Mettler-Toledo TGA/DSC3+ analyzer. Temperature and enthalpy adjustments were performed using indium, tin, and zinc, and then verified with indium. The balance was verified with calcium oxalate. The sample was placed in an aluminum pan. The pan was hermetically sealed, the lid pierced, and the pan was then inserted into the TG furnace. A weighed aluminum pan configured as the sample pan was placed on the reference platform. The furnace was heated under nitrogen. The sample was analyzed from 25° C. to 350° C. at 10° C./min.

Compound (3) Form A was found to have a melt onset at 82° C. (by DSC) and a glass transition was observed at −16° C. (ΔCₚ 0.3 J(g*K)). The lack of significant weight loss up to 220° C. by TGA is consistent with an anhydrous/unsolvated material. The steep drop in the TGA thermogram above 240° C. likely indicates decomposition.

A suitable crystal was culled and analyzed by single crystal X-ray diffraction (SCXRD). The crystal system is trigonal and the space group is R3c. The cell parameters and calculated volume are: a=27.7719(3) Å, b=27.7719(3) Å, c=7.94381(11) Å, α=90°, β=90°, γ=120°, V=5306.03(15) Å³. The molecular weight is 217.27 g mol-1 with Z=18, resulting in a calculated density of 1.224 g cm-3. Further details of the crystal data and crystallographic data collection parameters are summarized in Table 1.

TABLE 1
Crystal data and data collection parameters
for Compound (3) Form A

| | |
|---|---|
| Empirical formula | C₁₂H₁₅N₃O |
| Formula weight (g mol⁻¹) | 217.27 |
| Temperature (K) | 299.52(13) |
| Wavelength (Å) | 1.54184 |
| Crystal system | trigonal |
| Space group | R3c |
| Unit cell parameters | |
| a = 27.7719(3) Å | α = 90° |
| b = 27.7719(3) Å | β = 90° |
| c = 7.94381(11) Å | γ = 120° |
| Unit cell volume (Å³) | 5306.03(15) |
| Cell formula units, Z | 18 |
| Calculated density (g cm⁻³) | 1.224 |
| Absorption coefficient (mm⁻¹) | 0.648 |
| Goodness-of-fit on F² | S = 1.10 |
| Final residuals [ I > 2σ(I) ] | R = 0.0355, R_{w} = 0.0932 |

### Example 4: Stable Form and Polymorph Screen of Compound (3)

Approximate solubility values were measured for the as-received material at ambient temperature. The material exhibited high solubility in most of the organic solvents tested. Limited to intermediate solubility was observed in MTBE, toluene, water, and select solvent mixtures, while low solubility was observed only in heptane. The approximate solubility values were considered in the design of form screen experiments.

Stable form and polymorph screen experiments were conducted for compound (3), exploring a variety of solvent systems, temperatures, crystallization conditions, and starting materials. Techniques employed include slurrying, evaporation, cooling, antisolvent precipitation, vapor stressing, solid-state heating, milling, and lyophilization. Starting materials included compound (3) Form A as well as non-crystalline materials obtained from screening experiments. Temperatures spanning approximately −20° C. to 71° C. were explored. Aqueous and organic solvent systems were utilized, and select samples were analyzed while damp with solvent to screen for hydrates and solvates. No new forms were identified; compound (3) Form A or oily materials were obtained from all experiments.

Slurry experiments were conducted in an effort to identify the stable form at various conditions (Table 2). In these experiments, saturated solutions containing excess undissolved solids were stirred for extended durations. At these conditions, a metastable form would dissolve at a concentration that is supersaturated with respect to the stable form, causing crystallization of the more stable form over time. Slurries at room temperature (RT) and 2-8° C. were conducted for 2 weeks, while an elevated-temperature slurry was stirred for a shorter duration (3 days) to minimize the potential for decomposition. Solvent systems for which the compound was expected to exhibit limited or intermediate solubility were employed in an effort to provide suitable conditions for conversion to a more stable form, although the high solubility of compound (3) in most organic solvents limited the options. Compound (3) Form A was recovered from all slurries.

TABLE 2

| Solvent System (v/v) | Conditions^{a} | XRPD Result |
|---|---|---|
| Acetone/heptane | RT, 14 d | A (analyzed |
| 30:70 | | slightly damp) |
| DCM/cyclohexane | RT, 14 d | A (analyzed |
| 20:80 | | slightly damp) |
| heptane | 53° C., 3 d | A |
| MTBE | RT, 14 d | A (analyzed |
| | | slightly damp) |
| | 2-8° C., 14 d | A |
| toluene | RT, 14 d | A (analyzed damp) |
| | 2-8° C., 14 d | A |
| water | RT, 14 d | A (analyzed damp) |
| | 2-8° C., 14 d | A (analyzed damp) |

^{a}Times and temperatures are approximate.

In addition to slurry experiments, crystallization techniques exploring more kinetically driven conditions were utilized to screen for new forms (Table 3). While the majority of these experiments resulted in crystallization to compound (3) Form A, some evaporation experiments produced oily materials. Select oils were utilized for additional crystallization techniques to add variety to the starting materials for screening experiments. All of these experiments that showed signs of crystallization by microscopy were confirmed to be compound (3) Form A by XRPD. Some oils did not crystallize, particularly those involving aqueous solvent systems.

Experimental techniques used for the studies summarized in Table 3 were carried out as follows. In general, isolation of solids was done quickly after removing non-ambient samples from their respective temperature control devices to minimize equilibration to ambient temperature.

Decanting liquid phase: For some non-homogeneous slurries, solids were isolated by centrifuging the suspension (if needed) and discarding the liquid phase, leaving behind damp solids. Solids were dried briefly (e.g. air dried or under nitrogen) unless specified as “analyzed damp.”

Positive-pressure filtration: Solids were collected on 0.2-μm nylon or PTFE filters by pressing a slurry through a syringe and Swinnex filter holder assembly. In general, solids were dried briefly by blowing a 20-mL syringe of air over the filter several times. If designated as “analyzed damp,” solids were left damp with mother liquor. Some samples were additionally dried briefly under a gentle stream of nitrogen gas prior to analysis.

Vacuum filtration: Solids were collected on paper or nylon filters by vacuum filtration and air dried on the filters under reduced pressure briefly before transferring to a vial.

Fast evaporation: Solutions were prepared in various solvents and, typically, filtered through a 0.2-μm nylon or PTFE filter. Each solution was allowed to evaporate from an open vial at ambient conditions, unless otherwise stated. Solutions were allowed to evaporate to dryness unless designated as partial evaporations (solid present with a small amount of solvent remaining), in which case solids were isolated as described above.

Slow evaporation: Solutions were prepared in various solvents and, typically, filtered through a 0.2-μm nylon or PTFE filter. Each solution was allowed to evaporate from a covered vial (such as loosely capped or covered with perforated aluminum foil) at ambient conditions. Solutions were allowed to evaporate to dryness unless designated as partial evaporations (solids present with a small amount of solvent remaining), in which case solids were isolated as described above.

Vapor Stress: A small vial containing a given material was placed inside a larger vial containing solvent. The small vial was left uncapped, and the larger vial was capped to allow vapor stressing to occur at the stated temperature. Solids were isolated as described above.

Vapor Diffusion: Concentrated solutions were prepared in various solvents and, typically, filtered through a 0.2-μm nylon or PTFE filter. The filtered solution was dispensed into a small vial, which was then placed inside a larger vial containing antisolvent. The small vial was left uncapped, and the larger vial was capped to allow vapor diffusion to occur. Any solids present were isolated as described above.

Crash Precipitation: Solutions were prepared in various solvents and, typically, filtered through a 0.2-μm nylon or PTFE filter. Aliquots of various antisolvents were dispensed with stirring until precipitation occurred. Mixtures were allowed to stir for a specified amount of time. If necessary, samples were placed at sub-ambient temperatures to facilitate precipitation. Solids were isolated as described above.

Slow Cooling: Concentrated solutions were prepared in various solvents at an elevated temperature and, typically, filtered warm through a 0.2-μm nylon or PTFE filter into a warm vial. Each solution was capped and left on the hot plate, and the hot plate was turned off to allow the sample to slowly cool to ambient temperature. If no solids were present after cooling to ambient temperature, the sample was further cooled at subambient temperatures. Any solids present after cooling were isolated as described above.

Crash Cooling: Concentrated solutions were prepared in various solvents at an elevated temperature and, typically, filtered warm through a 0.2-μm nylon or PTFE filter into a warm vial. Each solution was capped and then immediately cooled to sub-ambient temperature, such as by placing in a freezer or plunging into a bath of dry ice and isopropanol. Solutions were allowed to remain at the sub-ambient temperature for a stated amount of time, and any solids present were isolated as described above.

Milling: Solids were transferred to an agate milling container. A small amount of solvent (if specified) and an agate milling ball were added to the container, which was then attached to a Retsch mill. The mixture was milled at the stated parameters, and the solids were scraped down the walls of the jar between cycles. The resulting solids were transferred to a clean vial and analyzed.

TABLE 3

| Solvent^{a} | Conditions^{b} | XRPD Result |
|---|---|---|
| — | heat solids at 71° C. for 1 h, tap | A |
| | periodically to redistribute solids | |
| — | mill at 30 Hz, | A |
| | 3 × 10 m | |
| acetone | FE | A |
| acetone/ | 1) add cyclohexane | A |
| cyclohexane | to acetone solution | |
| 1:4 | 2) SE | |
| acetone/water | SE | — |
| 50:50 | | |
| ACN | FE | — |
| DCM | FE | A |
| | SE | A |
| | 1) VS, RT, 1 d | A |
| | 2) remove from larger vial, | |
| | cap, stand in frz, 12 d | |
| | 3) SE in frz | |
| | VD w/ hexanes, 12 d | — |
| | mill at 30 Hz, 3 × 10 m | A |
| DCM | 1) CP w/ heptane at RT | A |
| DCM/heptane | 2) stir in frz, 13 d | |
| DCM | 1) CPw/MCHatRT | A |
| DCM/MCH | 2) stir in frz, 3 d | |
| EtOAc | SE | — |
| | CP w/ MCH at RT | A |
| EtOH | SE | — |
| EtOH/MCH | 1) add MCH to EtOH soln. | — |
| 1:4 | 2) FE | |
| IPA/MCH 1:4 | 1) add MCH to IPA soln. | A |
| | 2) stir in frz, l d | |
| | 3) attempt to filter | |
| | 4) FE | |
| IPOAc | FE | — |
| IPOAc | VD w/ hexanes, 4 d | A + minor peaks at |
| | | 11.7° and 20.5° |
| | | (analyzed damp) |
| IPOAc/ | 1) add heptane to | A |
| heptane 1:2 | IPOAc soln. | |
| | 2) stir in frz, 2 d | |
| | 3) filter while cold | |
| MEK | FE | A |
| | VD w/ hexanes, 4 d | A (analyzed damp) |
| MeOH | FE | — |
| MeOH/water | SE | — |
| 50:50 | | |
| MTBE | VS, RT, 8 d | A |
| THF/water | SE | — |
| 50:50 | | |
| toluene | VS, RT, 8 d | A |
| | SC, 53-56° C. to | A (analyzed damp) |
| | RT, stand at RT l d | |
| | 1) CC, 53-56° C. | A |
| | to frz, stand in frz 4 d | |
| | 2) CP w/ hexanes | |
| water | VS, RT, 8 d | A |
| | 1) SC, 53-56° C. to | — |
| | RT, stand at RT 1 d | |
| | 2) FE | |
| | CC, 53-56° C. to | — |
| | ref, stand in ref 15 d | |

^{a}Solvent ratios are v/v.
^{b}Times and temperatures are approximate.
FE = fast evaporation;
SE = slow evaporation;
VS = vapor stress;
VD = vapor diffusion;
CP: crash precipitation;
SC = slow cooling;
CC = crash cooling.
ACN = acetonitrile;
EtOAc = ethyl acetate;
EtOH = ethanol;
MCH = Methylcyclohexane;
IPA = isopropyl alcohol;
IPOAc = isopropyl acetate;
MEK = methyl ethyl ketone;
MeOH = methanol.
frz = freezer.

### Example 5: Preparation of Compound (3)

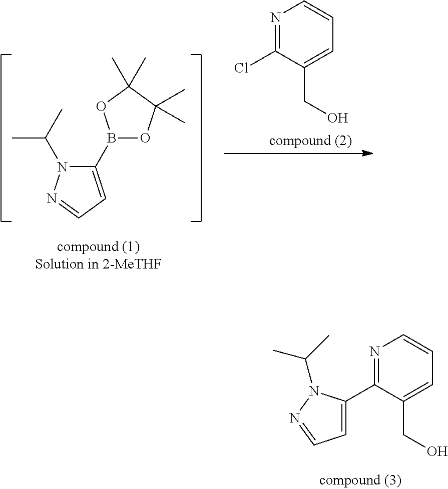

A mixture of compound (2) (10.0 g), Pd(Amphos)₂Cl₂ (15 mg), sodium bicarbonate (14.7 g), and 2-MeTHF solution containing compound (1) (2.13 g) and 2-MeTHF (about 80 mL) are degassed and heated to about 70 to about 80° C. for longer than 10 hours. The resulting mixture is washed with 10% brine (50 g), and the organic solution is diluted with heptane (40 mL). The combined organic solution is azeotropically dried by distillation and with heptane/MeTHF as chase solvent. The resulting mixture is passed through charcoal and diluted with heptane. Additional heptane is added, and the slurry is cooled to 0-20° C. and filtered to provide compound (3).

### Example 6: Preparation of a Bishydrochloric Acid Salt of Compound (4)

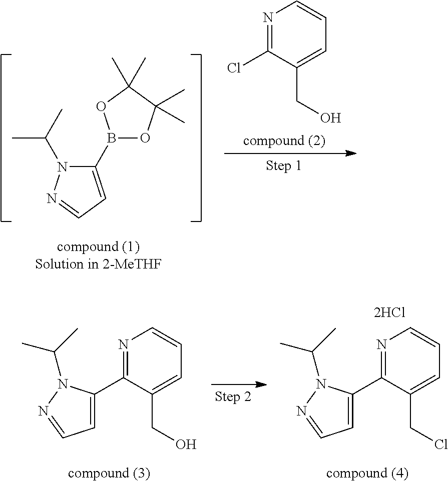

Step 1: A mixture of compound (2) (15.0 g), Pd(Amphos)₂Cl₂ (222 mg), sodium bicarbonate (21.9 g), and 2-MeTHF solution containing compound (1) (32.07 g) and 2-MeTHF (about 120 mL) were degassed and heated to about 70 to about 80° C. for longer than 10 hours. The resulting mixture was washed with 10% brine (75 g), and the organic solution was diluted with heptane (105 mL). The combined organic solution was azeotropically dried by distillation and with heptane (45 mL)/MeTHF (15 mL) as chase solvent. To the resulting mixture (˜75 mL) was charged MeTHF (15 mL), then passed through charcoal and diluted with heptane (105 mL). The resulting slurry was cooled to 0-20° C. and filtered to provide compound (3) (72% yield).

Step 2: To a solution of SOCl₂ (10.4 g) in dichloromethane (DCM) (30 mL) was added a solution of compound (3) (10.0 g) in dichloromethane (DCM) (47 mL). After the reaction met IPC, water (0.7 g) was subsequently added to achieve a bishydrochloric acid salt of compound (4) (99% yield).

### Example 7: Preparation of a Bishydrochloric Acid Salt of Compound (4)

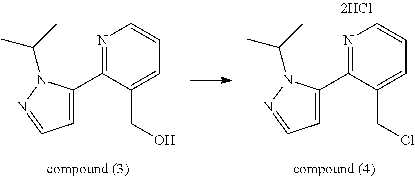 [SMILES: CC(C)n1nccc1-c1ncccc1CO ; CC(C)n1nccc1-c1ncccc1CCl]

### Studies on Addition Sequence

The impact on yield from the order of addition of base, solvent, and chlorinating agent for conversion of a hydrochloric acid salt of compound (3) to a bishydrochloric acid salt of compound (4) was studied. To a hydrochloric acid salt of compound (3) was added dichloromethane (DCM) first, followed by sodium bicarbonate, or was added sodium bicarbonate first, followed by DCM (Table 4). Thionyl chloride was subsequently added to the organic layer to achieve a bishydrochloric acid salt of compound (4). The results are summarized in Table 4.

TABLE 4

| Amount (in kg) of | | Yield of a |
|---|---|---|
| a hydrochloric | | bishydrochloric |
| acid salt of | Mode of | acid salt |
| compound (3) | addition | compound (4) |
| 414.6 | A then C | 89% |
| 398.9 | B then C | 95% |
| 379.0 | B then C | 90% |
| 389.5 | B then C | 90% |
| 375.7 | B then C | 92% |

A: charge aqueous sodium bicarbonate, then DCM;
B: charge DCM, then aqueous sodium bicarbonate;
C: charge thionyl chloride.

### Addition of DCM and Aqueous Sodium Bicarbonate, Followed by Thionyl Chloride

A hydrochloric acid salt of compound (3) (1 equivalent) and water were charged to a reactor A, and the contents were agitated at about 15° C. until a clear solution formed. The contents of the reactor were filtered through charcoal into a clean reactor B. The filtrate was combined with DCM. With agitation, aqueous sodium bicarbonate (1.2 equivalents) was charged to reactor B while maintaining temperature at about 20° C. The contents of the reactor B were settled, and the bottom organic layer was transferred to a clean reactor C. DCM was charged to the reactor B, and the contents agitated while maintaining temperature. The contents of the reactor B were settled, and the bottom layer was transferred to the reactor C. The upper aqueous layer was discarded.

The contents of the reactor C were concentrated under atmospheric pressure until the IPC criterion for water content was met. The contents of the reactor C were slowly charged via a polish filter to reactor D containing SOCl₂ (1.9 equivalents) in DCM. Seeding with a bishydrochloric acid salt of compound (4), which can be prepared as described herein, can be performed in the middle of the addition. The contents of the reactor D were agitated at about 20° C. until IPC criterion for reaction completion was met.

Then water was slowly charged sub-surface to the reactor while maintaining temperature. The contents of the reactor D were further agitated while maintaining temperature. The contents of the reactor were isolated and the cake was washed with DCM. The cake was dried until IPC criterion for residual solvent and chloride content were met, providing compound (4) as a bishydrochloride acid salt.

A bishydrochloric acid salt of compound (4) prepared via this method provides a product with high purity and yield and also can be converted to a compound of Formula (I) with high purity and yield.

### Example 8: Preparation of the Compound of Formula (I)

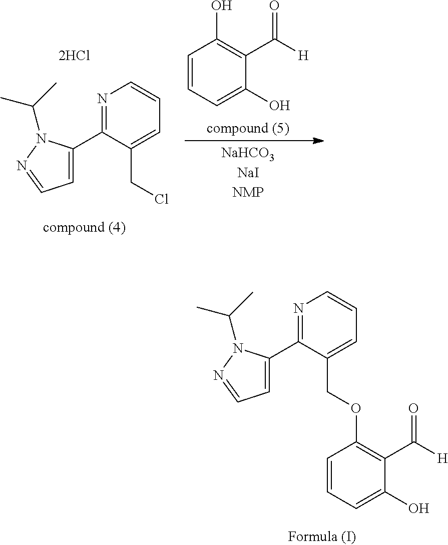

A bishydrochloride salt of compound (4) (1 equivalents) and NMP were charged into a reactor, and the contents were agitated until solids have dissolved. The contents of the reactor were filtered through charcoal into a clean reactor. Sodium bicarbonate (3.2 equivalents) was slowly charged to the reactor while maintaining temperature at about 20° C. Sodium iodide (about 1 equivalent) and compound (5) (about 1.2 equivalents) were charged to the reactor, and the contents were heated to about 50° C. and agitated until reaction completion. Water and a compound of formula (I) Form I seeds, which were prepared according to known methods such as those described in U.S. Pat. No. 9,447,071, were charged to the reactor while maintaining temperature of about 45° C. Water was slowly charged to the reactor while maintaining temperature. The contents of the reactor were cooled to about 20° C. and agitated while maintaining temperature. The contents of the reactor were isolated, and the cake was washed with NMP and water. The cake was dried under reduced pressure, providing a compound of Formula (I).

10% brine may be used as an antisolvent instead of water during the precipitation of crude compound of Formula (I) and provides improved yields of compound of Formula (I) as summarized in Table 5.

TABLE 5

| | | Formula (I) | |
|---|---|---|---|
| Process | Scale | % Purity | % Yield |
| Water as | 300-350 kg | 99.9-100.0 | 65-72% |
| anti-solvent | | | |
| 10% brine as | 0.5 kg | 100.0% | 76% |
| anti-solvent | | | |

Form II of a compound of Formula (I) can be achieved as follows. A compound of Formula (I) and MTBE were charged to a reactor, and the contents were heated to about 30° C. and agitated while maintaining temperature. Filter aid was charged to the reactor, and the contents were cooled and agitated. The contents of the reactor were filtered into a clean reactor. Water was charged to the reactor, and the contents were agitated while maintaining temperature. The contents of the reactor were settled, and the lower aqueous layer was removed. Water was charged to the reactor, and the contents were agitated while maintaining temperature. The contents of the reactor were settled, and the lower aqueous layer was removed. The aqueous layers were discarded. The contents of the reactor were concentrated under atmospheric pressure. The contents of the reactor were filtered into a clean reactor, and the volume was reduced by atmospheric distillation. The contents of the reactor were heated to about 45-55° C. and n-Heptane and Form II of a compound of formula (I) seeds, which were prepared according to known methods such as those described in U.S. Pat. No. 9,447,071, were charged, and the contents were agitated while maintaining temperature. N-Heptane was slowly added to the reactor, and the contents were agitated while maintaining temperature. The contents of the reactor were cooled to about 3° C. and agitated while maintaining temperature. The contents of the reactor were isolated, and the cake was washed with n-heptane. The cake was dried under reduced pressure, providing Form II of a compound of Formula (I).

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

All publications, patent applications, patents, and other references mentioned herein are expressly incorporated by reference in their entirety, to the same extent as if each were incorporated by reference individually. In case of conflict, the present specification, including definitions, will control.

What is claimed:

1. A process for preparing a compound of Formula (I) 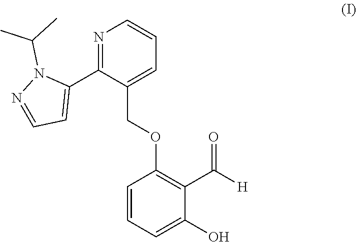 [SMILES: CC(C)n1nccc1-c1ncccc1COc1cccc(O)c1C=O]
comprising:
(i) contacting compound (1) 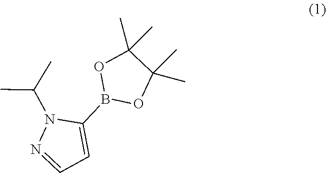 [SMILES: CC(C)n1nccc1B1OC(C)(C)C(C)(C)O1]
with compound (2) 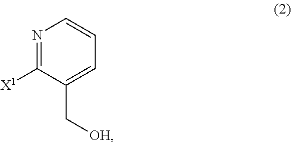
wherein X¹ is Cl, Br, I, or triflate (OTf),
in the presence of a catalyst of formula (7) 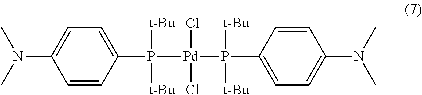
under reaction conditions sufficient to form compound (3) 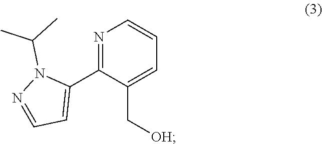 [SMILES: CC(C)n1nccc1-c1ncccc1CO]
(ii) forming a salt of compound (3);
(iii) contacting the salt of compound (3) with a base, an organic solvent, and a chlorinating agent under reaction conditions sufficient to form compound (4) 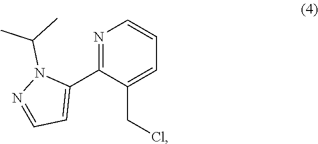 [SMILES: CC(C)n1nccc1-c1ncccc1CCl]
or a salt thereof; and
(iv) contacting compound (4), or a salt thereof, with compound (5) 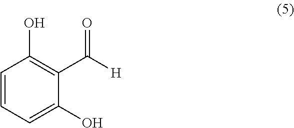
under reaction conditions sufficient to form the compound of Formula (I); and
wherein step (iii) results in substantially no formation of a solid form of compound (3).

2. A process for preparing a compound of Formula (I) 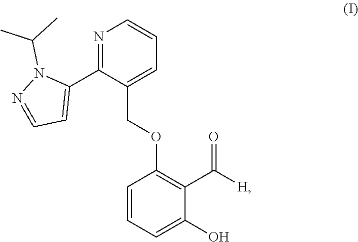
comprising:
(i) contacting compound (1) 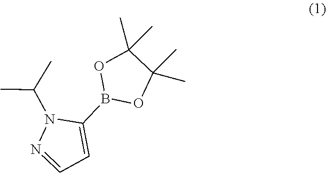 [SMILES: CC(C)n1nccc1B1OC(C)(C)C(C)(C)O1]
with compound (2) 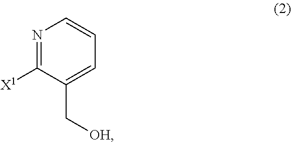
wherein X¹ is Cl, Br, I, or triflate (OTf),
in the presence of a catalyst of formula (7) 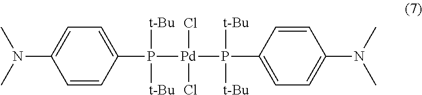
under reaction conditions sufficient to form compound (3) 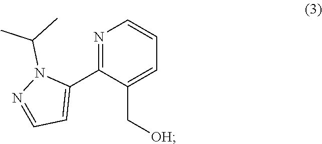 [SMILES: CC(C)n1nccc1-c1ncccc1CO]
(ii) forming a salt of compound (3);
(iii) contacting the salt of compound (3) with an organic solvent to form a mixture;
(iv) contacting the mixture with a base to form a solution of compound (3);
(v) contacting the solution of compound (3) with a chlorinating agent under reaction conditions sufficient to form compound (4) 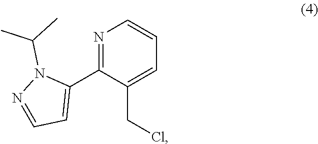 [SMILES: CC(C)n1nccc1-c1ncccc1CCl]
or a salt thereof; and
(vi) contacting compound (4), or a salt thereof, with compound (5) 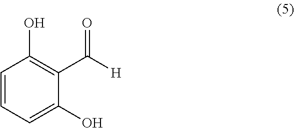
under reaction conditions sufficient to form the compound of Formula (I).

3. A process for preparing a compound of Formula (I) 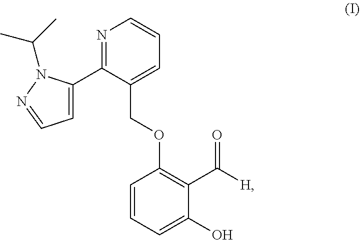
comprising:
(i) contacting compound (1) 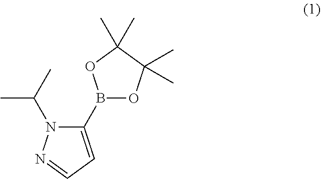 [SMILES: CC(C)n1nccc1B1OC(C)(C)C(C)(C)O1]
with compound (2) 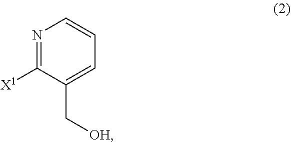
wherein X¹ is Cl, Br, I, or triflate (OTf),
in the presence of a catalyst of formula (7) 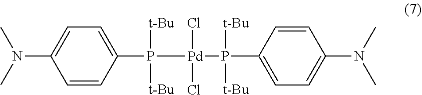
under reaction conditions sufficient to form compound (3) 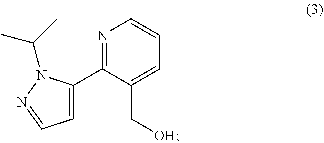 [SMILES: CC(C)n1nccc1-c1ncccc1CO]
(ii) contacting compound (3) with an organic solvent and a chlorinating agent under reaction conditions sufficient to form compound (4) 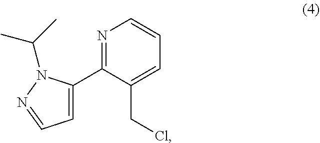 [SMILES: CC(C)n1nccc1-c1ncccc1CCl]
or a salt thereof; and
(iii) contacting compound (4), or a salt thereof, with compound (5) 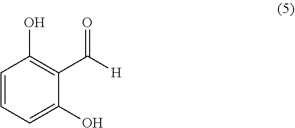
under reaction conditions sufficient to form the compound of Formula (I).

4. A process for preparing a compound of Formula (I) 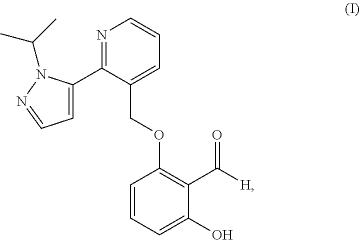
comprising
(i) contacting about 1.3 equivalents of compound (1) 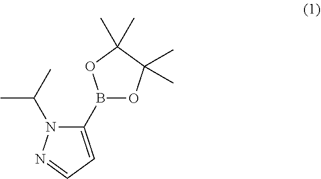 [SMILES: CC(C)n1nccc1B1OC(C)(C)C(C)(C)O1]
with 1 equivalent of compound (2) 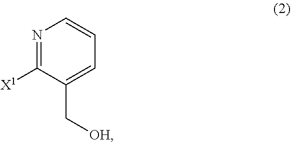
wherein X¹ is Cl, Br, I, or triflate (OTf),
in the presence of about 0.003 equivalents of a catalyst of formula (7) 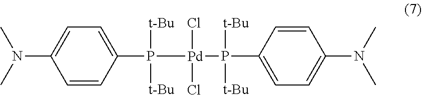
relative to equivalents of compound (2),
and about 2.5 equivalents of sodium bicarbonate relative to equivalents of compound (2),
in 2-methyltetrahydrofuran as a solvent,
at a temperature of about 70° C. to about 80° C. to form compound (3) 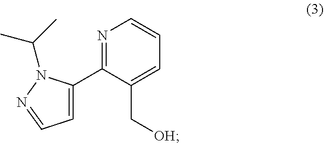 [SMILES: CC(C)n1nccc1-c1ncccc1CO]
(ii) forming a salt of compound (3);
(iii) contacting the salt of compound (3) with a base, an organic solvent, and a chlorinating agent under reaction conditions sufficient to form compound (4) 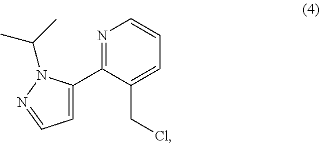 [SMILES: CC(C)n1nccc1-c1ncccc1CCl]
or a salt thereof; and
(iv) contacting compound (4), or a salt thereof, with compound (5) 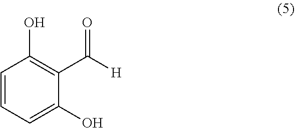
under reaction conditions sufficient to form the compound of Formula (I).

5. The process of any one of claims 2-4, wherein compound (3) is isolated in a crystalline form characterized by an X-ray powder diffractogram comprising the following peaks: 14.79°, 22.67°, and 24.44°2θ, each ±0.2°2θ, as determined on a diffractometer using Cu-Kα radiation.

6. The process of claim 5, wherein the diffractogram further comprises peaks at 11.02°, 16.88°, 17.34°, and 26.09°2θ, each ±0.2°2θ.

7. The process of any one of claims 1-2 and 4, wherein the salt of compound (3) is a hydrochloric acid salt.

8. The process of claim 7, wherein forming a salt of compound (3) comprises contacting compound (3) with HCl.

9. The process of any one of claims 1-8, wherein the salt of compound (4) is a hydrochloric acid salt.

10. The process of any one of claims 1-9, wherein X¹ is Cl or Br.

11. The process of any one of claims 1-10, wherein X¹ is Cl.

12. The process of any one of claims 1-3, wherein the reaction conditions in step (i) comprise 2-methyltetrahydrofuran as a solvent.

13. The process of any one of claims 1-3, wherein the reaction conditions in step (i) comprise a temperature of about 70° C. to about 80° C.

14. The process of any one of claims 1-3 and 12-13, wherein the reaction conditions in step (i) comprise about 2.5 equivalents of a base relative to equivalents of compound (2).

15. The process of claim 14, wherein the base of step (i) is sodium bicarbonate.

16. The process of any one of claims 1-3 and 12-15, wherein the reaction conditions in step (i) comprise about 0.001 equivalents to about 0.005 equivalents of the catalyst relative to equivalents of compound (2).

17. The process of any one of claims 1-3 and 12-16, wherein the reaction conditions in step (i) comprise about 0.003 equivalents of the catalyst relative to equivalents of compound (2).

18. The process of claim 4, wherein step (i) is performed at a temperature of about 75° C.

19. The process of any one of claims 1 or 4, wherein the base of step (iii) comprises sodium bicarbonate, the organic solvent of step (iii) comprises dichloromethane, and the chlorinating agent of step (iii) comprises SOCl₂.

20. The process of claim 2, wherein the organic solvent of step (iii) comprises dichloromethane, the base of step (iv) comprises sodium bicarbonate, and the chlorinating agent of step (v) comprises SOCl₂.

21. The process of claim 3, wherein the organic solvent of step (ii) comprises dichloromethane, and the chlorinating agent of step (ii) comprises SOCl₂.

22. The process of any one of claims 1 or 4, wherein reaction conditions in step (iv) comprise N-methyl-2-pyrrolidone (NMP), sodium bicarbonate, and NaI.

23. The process of claim 2, wherein reaction conditions in step (vi) comprise N-methyl-2-pyrrolidone (NMP), sodium bicarbonate, and NaI.

24. The process of claim 3, wherein reaction conditions in step (iii) comprise N-methyl-2-pyrrolidone (NMP), sodium bicarbonate, and NaI.

25. The process of any one of the preceding claims, further comprising isolating the compound of Formula (I) by adding 10% brine as an antisolvent.

26. The process of any one of claims 1-25, further comprising crystallizing the compound of Formula (I) to obtain a crystalline ansolvate of the compound of Formula (I) characterized by an X-ray powder diffractogram comprising the following peaks: 13.37°, 14.37°, 19.95° and 23.92 °2θ, each ±0.2° 2θ, as determined on a diffractometer using Cu-Kα radiation.

27. The process of claim 26, wherein crystallizing comprises contacting the compound of Formula (I) with methyl tert-butyl ether and n-heptane.

28. A crystalline form of compound (3) 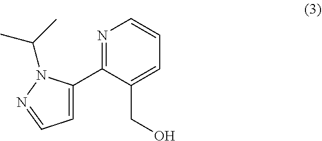 [SMILES: CC(C)n1nccc1-c1ncccc1CO]
characterized by an X-ray powder diffractogram comprising the following peaks: 14.79°, 22.67°, and 24.44° 2θ, each ±0.2° 2θ, as determined on a diffractometer using Cu-Kα radiation.

29. The crystalline form of claim 28, wherein the diffractogram further comprises peaks at 11.02°, 16.88°, 17.34°, and 26.09° 2θ, each ±0.2° 2θ.

30. The crystalline form of any one of claims 28-29, characterized by an X-ray powder diffractogram as substantially shown in FIG. 3.

31. The crystalline form of any one of claims 28-30, characterized by a differential scanning calorimetry (DSC) curve comprising an endotherm at about 82° C. (onset temperature).

32. The crystalline form of any one of claims 28-31, characterized by a DSC curve as substantially shown in FIG. 4.
